(19) United States
(12) Patent Application Publication
(10) Pub. No.: US 2012/0157860 A1
(43) Pub. Date: Jun. 21, 2012

(54) PULSE MEASURING DEVICE AND METHOD

(75) Inventor: Takuji Suzuki, Kanagawa (JP)

(73) Assignee: KABUSHIKI KAISHA TOSHIBA, Tokyo (JP)

(21) Appl. No.: 13/407,002

(22) Filed: Feb. 28, 2012

Related U.S. Application Data
(63) Continuation of application No. PCT/JP2009/065351, filed on Sep. 2, 2009.

(51) Int. Cl. A61B 5/0205

(52) U.S. Cl. 600/484

(57) ABSTRACT

According to an embodiment, a pulse measuring device includes a pulse detecting unit, an amplitude calculating unit, an interval calculating unit, an extracting unit, and a determining unit. The pulse detecting unit is configured to detect a pulse of a subject. The amplitude calculating unit is configured to calculate an amplitude of the pulse. The interval calculating unit is configured to calculate an interval of the pulse. The extracting unit is configured to extract a fluctuation variation component which varies in operative association with breathing of the subject from the interval. The determining unit is configured to determine that an apnea event occurs when the amplitude or the interval is reduced at a first time and the fluctuation variation component is reduced at a second time which is immediately before the first time.

### CROSS-REFERENCE TO RELATED APPLICATIONS

This application is a continuation of PCT international application Ser. No. PCT/JP2009/065351 filed on Sep. 2, 2009 which designates the United States; the entire contents of which are incorporated herein by reference.

### FIELD

The invention relates to measurement of a pulse.

### BACKGROUND

Various devices for examining sleep apnea syndrome are known. In such devices, a period from stop of breathing to start of breathing again is defined as an event of apnea (or, an apnea event). Counting the number of apnea events is used to examine the sleep apnea syndrome. For example, a device is known that determines apnea from a reduction in amplitude. Also, a device is known that detects breath abnormality from the ratio of the amplitude of a pulse to the number of pulses. Furthermore, a device is known that detects low cycle variation due to apnea.

However, in the related art, it is difficult to discriminate the reaction of a subject due to factors other than the sleep apnea syndrome, such as a body motion, from the reaction due to the sleep apnea syndrome.

### BRIEF DESCRIPTION OF THE DRAWINGS

FIG. 1 is a diagram illustrating a structure of a pulse measuring device.

FIG. 2 is a diagram illustrating a fluctuation variation component calculating process.

FIG. 3 is a diagram illustrating the number of pulses, an amplitude of a pulse, and a fluctuation variation component when an apnea event occurs.

FIG. 4 is a flowchart illustrating a pulse measuring process.

FIG. 5 is a diagram illustrating a pulse amplitude and pulse interval calculating process.

FIG. 6 is a diagram illustrating the number of pulses, the amplitude of a pulse, and the fluctuation variation component when an apnea event and a hypopnea event occur.

FIG. 7 is a diagram illustrating the structure of a pulse measuring device.

### DETAILED DESCRIPTION

According to an embodiment, a pulse measuring device includes: a pulse detecting unit, an amplitude calculating unit, an interval calculating unit, an extracting unit, and a determining unit. The pulse detecting unit is configured to detect a pulse of a subject. The amplitude calculating unit is configured to calculate an amplitude of the pulse. The interval calculating unit is configured to calculate an interval of the pulse. The extracting unit is configured to extract a fluctuation variation component which varies in operative association with breathing of the subject from the interval. The determining unit is configured to determine that an apnea event occurs when the amplitude or the interval is reduced at a first time and the fluctuation variation component is reduced at a second time which is immediately before the first time.

Hereinafter, various embodiments will be described in detail with reference to the accompanying drawings.

In a pulse measuring device according to a first embodiment, a sensor (that detects a pulse as biological information of a subject (user, or person being tested)) calculates a pulse interval, detects an apnea event, counts the number of apnea events per hour, and stores the number counted.

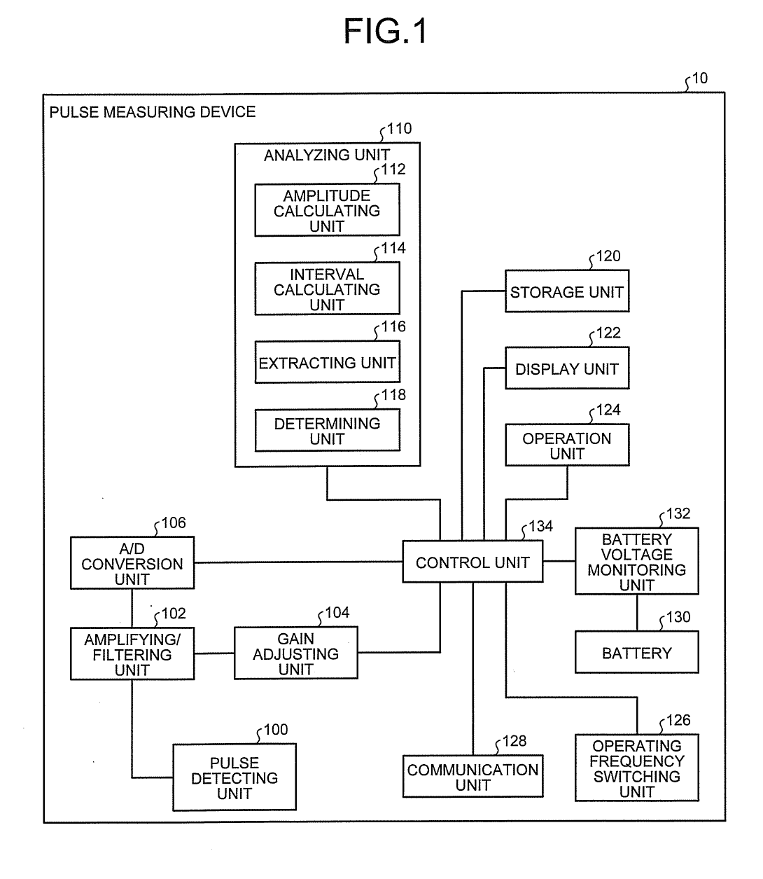

As illustrated in FIG. 1, a pulse measuring device 10 includes a pulse detecting unit 100, an amplifying/filtering unit 102, a gain adjusting unit 104, an A/D (analog/digital) conversion unit 106, an analyzing unit 110, a storage unit 120, a display unit 122, an operation unit 124, an operating frequency switching unit 126, a communication unit 128, a battery 130, a battery voltage monitoring unit 132, and a control unit 134. The analyzing unit 110 includes an amplitude calculating unit 112, an interval calculating unit 114, an extracting unit 116, and a determining unit 118. The pulse measuring device can be configured in, for example, a wristwatch shape so as to be worn on the wrist when used.

The pulse detecting unit 100 is provided on the lower surface of the pulse measuring device 10 and measures the subject's pulse. The pulse detecting unit 100 includes a green LED and a photodiode and measures the subject's pulse with the photodiode, by emitting light from the green LED onto the skin surface of the wrist and by detecting the variation in reflected light which is caused by a change in the blood flow of the capillaries.

The amplifying/filtering unit 102 amplifies and filters the waveform of the pulse measured by the pulse detecting unit 100. Specifically, the amplifying/filtering unit 102 converts a current that is output from the photodiode of the pulse detecting unit 100 into a voltage with a current-voltage converter and amplifies the voltage with an amplifier, thereby implementing the functions of a high-pass filter (for example, a cutoff frequency: 0.1 Hz) and a low-pass filter (for example, a cutoff frequency: 50 Hz).

The gain adjusting unit 104 adjusts the gain of the amplifying/filtering unit 102 according to the measured state. Specifically, the gain adjusting unit 104 calculates the amplitude of the pulse input to the control unit 134 and controls the gain of the amplifying/filtering unit 102 from the relation between the amplitude and a set threshold value. The A/D conversion unit 106 performs A/D conversion on the output of the pulse detecting unit 100.

The analyzing unit 110 acquires the pulse waveform which has been detected by the pulse detecting unit 100, amplified and filtered by the amplifying/filtering unit 102, and subjected to A/D conversion by the A/D conversion unit 106, and analyzes the acquired pulse waveform. The amplitude calculating unit 112 of the analyzing unit 110 calculates a pulse amplitude, which is the amplitude of the pulse on the basis of the pulse waveform. The interval calculating unit 114 calculates a pulse interval, which is the interval between the pulses on the basis of the pulse waveform.

The extracting unit 116 extracts a fluctuation variation component which is included in the pulse interval and varies in operative association with the breathing of the subject. In this embodiment, value of aMSSD is calculated as the fluctuation variation component.

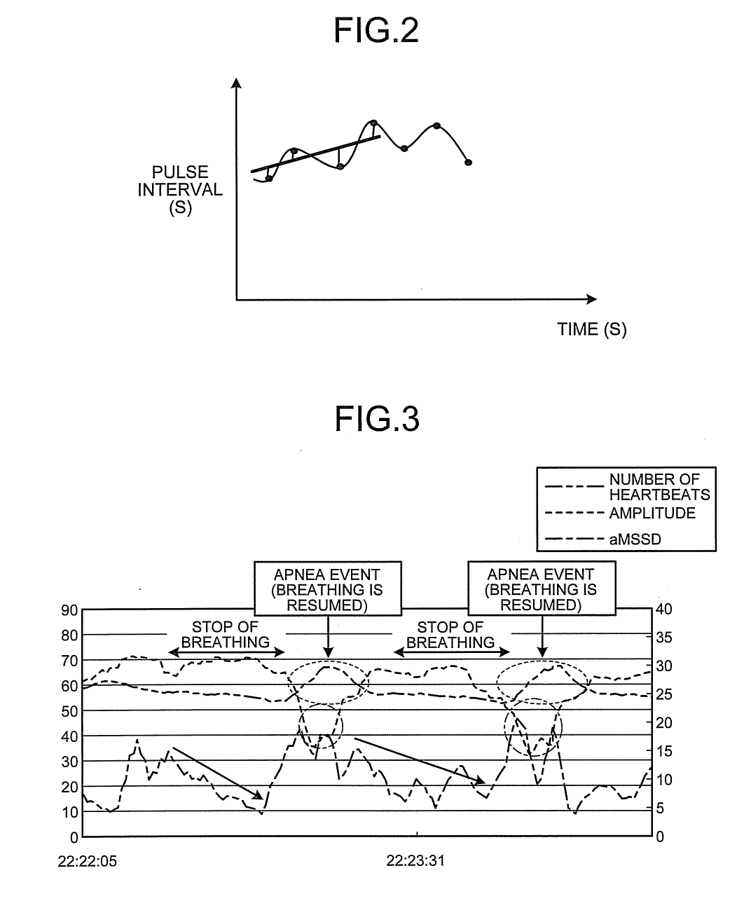

Next, the calculation of aMSSD will be described. An apnea event occurs at an interval of several tens of seconds. Therefore, it is difficult to calculate the fluctuation variation component at the pulse interval using the general frequency analysis method according to the related art, such as FFT. In this embodiment, it is assumed that a time axis analysis method is used. Specifically, as illustrated in FIG. 2, linear regression is used on the pulse interval of the set number of pulses (for example, four pulses) sufficiently including one period of breathing. Then, the sum of squares of the difference between an estimated value and the observed pulse interval is calculated (,which is called aMSSD). In this way, the influence of a frequency component which is slower than the period of breathing can be removed and the fluctuation variation component can be extracted. This calculation is performed for each pulse to check small variations on the time axis. Specifically, aMSSD is calculated by the following Expression (1):

$$aMSSD = {\sum }_{n = 1}^{4} {( RR ( n ) - R \hat{R} ( n ) )}^{2} / 4 \qquad ( 1 )$$

In Expression (1), the first term is the value of the observed pulse interval and the second term is the value of the estimated pulse interval.

In this embodiment, it is assumed that aMSSD obtained by Expression (1) is a fluctuation variation component at the time when a pulse interval RR(4) is obtained. As another example, aMSSD obtained by Expression (1) may be a fluctuation variation component at the time when a pulse interval RR(1) is obtained. As such, aMSSD at a given time may be calculated by a plurality of pulse intervals including the pulse interval at that time.

The determining unit 118 determines whether an apnea event occurs on the basis of the fluctuation variation component at any one of the pulse amplitudes or the pulse intervals. FIG. 3 is a graph illustrating a variation in the actually measured values of the number of pulses (60×reciprocal of the pulse interval), the pulse amplitude, and the fluctuation variation component over time. As such, when an apnea event occurs (which means breathing is resumed), the number of pulses increase (the pulse interval is reduced) and the pulse amplitude is reduced. In addition, the fluctuation variation component is gradually reduced before breathing is resumed and increases at the same time as breathing is resumed.

The determining unit 118 determines that an apnea event occurs when the pulse interval is reduced or the pulse amplitude is reduced at a given time (hereinafter, referred to as a first time) and the fluctuation variation component is reduced at the time immediately before the given time (hereinafter, referred to as a second time). The second time is earlier than the first time and a time length between the first time and the second time is less than the period of the apnea event.

The storage unit 120 is a memory that stores various kinds of data including measurement data, such as the pulse interval, the pulse amplitude, the fluctuation variation component which is the analysis result of the analyzing unit 110, and threshold values which will be described below. The storage unit 120 may be of, for example, a flash memory.

The display unit 122 is a display device that displays time, the number of pulses, a pulse measured state, a battery state, a memory state, and a communication state. The display unit 122 may be, for example, a liquid crystal display (LCD).

The operation unit 124 includes, for example, a mode switch for switching between the time mode and the measurement mode or a push switch for turning on a backlight. The operating frequency switching unit 126 changes the operating frequency according to the set mode. In the time mode, the operating frequency switching unit 126 changes the operating frequency to the minimum value required to manage time, thereby reducing power consumption.

The communication unit 128 performs data communication with external apparatuses, such as a PC, a PDA terminal, and a mobile phone and may be, for example, a USB. In this way, the communication unit 128 can measure and accumulate data during sleep for a plurality of days, be connected to a USB port of a PC, store the data in a format which can be analyzed by predetermined analysis software in a hard disk of the PC for example, and analyze the data using analysis software. The battery 130 supplies power to the entire pulse measuring device 10. The battery voltage monitoring unit 132 monitors the voltage of the battery 130.

The control unit 134 is a controller that controls the overall operation of the pulse measuring device 10 and controls the flow of data and processing requests related to each processing unit by receiving requests and instructions from the subject. Specifically, the control unit 134 controls, for example, the turning-on/turning-off of a power supply, the start of measurement, and various kinds of processes related to measurement in response to requests from the subject.

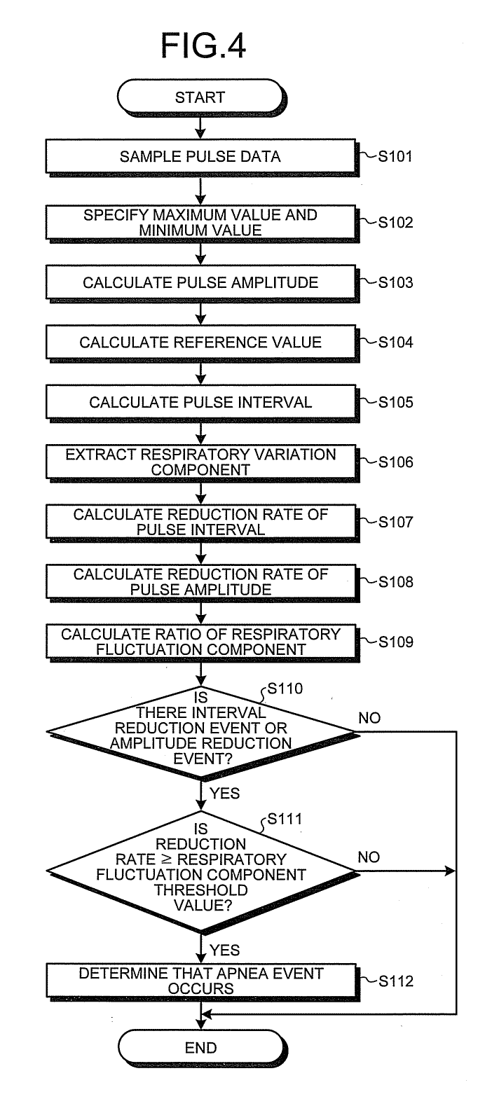

Next, a pulse measuring process of the pulse measuring device 10 will be described. As illustrated in FIG. 4, first, the pulse detecting unit 100 samples pulse data (Step S101). Then, the amplitude calculating unit 112 specifies the maximum value and the minimum value of the pulse data for about one second before and after the processing point of a series of sampled pulse data (Step S102).

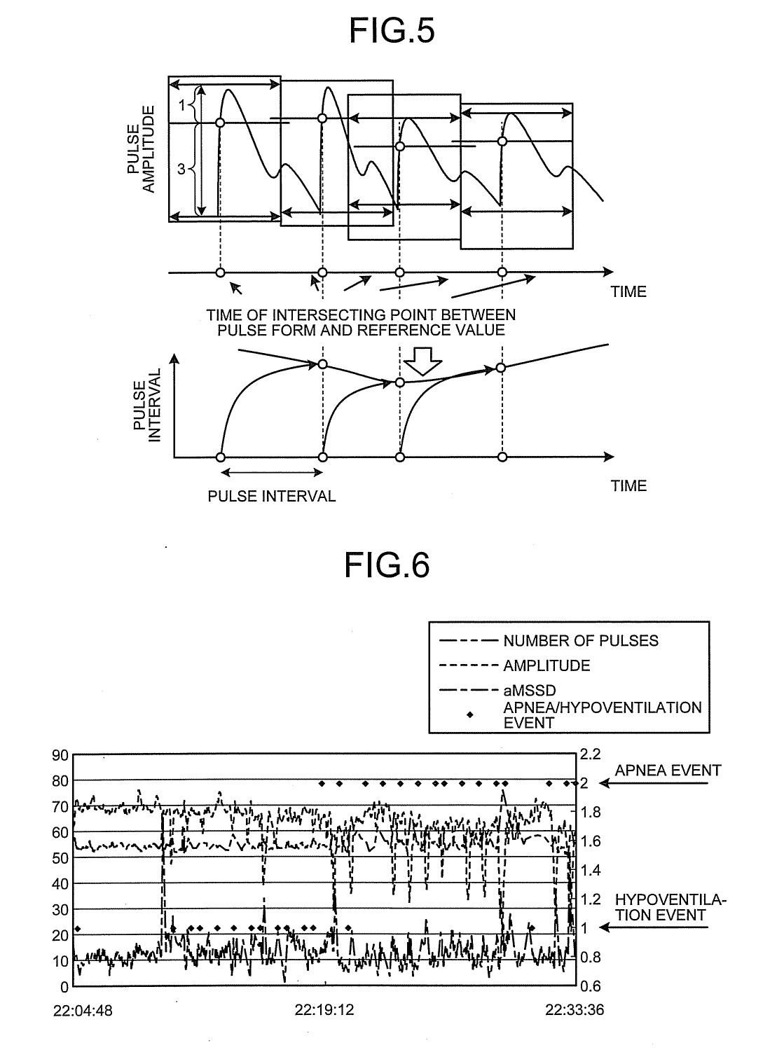

Then, the amplitude calculating unit 112 calculates the pulse amplitude from the maximum value and the minimum value. For example, as illustrated on the upper side of FIG. 5, the amplitude calculating unit 112 calculates the difference between the maximum value and the minimum value as the pulse amplitude (Step S103). Then, the amplitude calculating unit 112 calculates an internally dividing point (for example, 3:1) of the calculated pulse amplitude as a reference value (Step S104).

Then, as illustrated on the lower side of FIG. 5, the interval calculating unit 114 calculates the time of an intersection point between the pulse waveform and the reference value from a series of pulse data from which a DC (direct current) variation component is removed, and calculates the time interval between the calculated time and the previous intersection point as the pulse interval (Step S105).

The extracting unit 116 extracts the fluctuation variation component on the basis of the pulse interval using Expression (1) (Step S106). The pulse amplitude, the pulse interval, and the fluctuation variation component calculated at each point of time are stored in the storage unit 120, being associated with the detection time.

Then, the determining unit 118 calculates the reduction rate of the pulse interval (Step S107). Specifically, the determining unit 118 first compares an average interval with each of a plurality of pulse intervals obtained in a time window including the first time, which is a processing target. The time window is the time range (hereinafter, referred to as a first time range) of a predetermined time length and is set to be, for example, 30 seconds. The average interval is the average value of the plurality of pulse intervals obtained in a predetermined time range (hereinafter, referred to as a second time range) before the time window. The second time range is set to be, for example, 1 minute.

Then, the determining unit 118 counts the number of pulse intervals in which the reduction rate determined by an interval difference, which is the difference between the two intervals, is equal to or larger than a predetermined interval threshold value. In this embodiment, it is assumed that the interval threshold value is, for example, 0.1. The reduction rate is obtained by the following Expression (2):

Reduction Rate=(Average Interval−Pulse Interval in Time Window)/Average Interval (2)

The determining unit 118 calculates the ratio of the number of counted pulse intervals in all the pulse intervals in the time window. When the ratio of the number of pulse intervals is equal to or larger than a first predetermined number threshold value, the determining unit 118 determines that there is a pulse interval reduction event. The first number threshold value indicates the ratio.

As described above, it is known that the pulse interval is reduced (the number of pulses increases) when breathing is resumed during an apnea event. As such, it is possible to specify the resumption time of breathing by checking for the reduction of the pulse interval.

As another example, when the counted number of pulse intervals, not the ratio of the number of pulse intervals, is equal to or larger than the first number threshold value, the determining unit may determine that there is a pulse interval reduction event. In this case, the first number threshold value indicates the number of pulse intervals.

As another example, the determining unit may count the number of pulse intervals in which the interval difference is equal to or larger than a predetermined interval threshold value, instead of calculating the reduction rate, and may determine whether there is a reduction event on the basis of the number of pulse intervals.

As another example, the determining unit may determine whether there is a reduction event on the basis of the counted number of successive pulse intervals which are greater than the interval threshold value. That is, when the number of successive pulse intervals that are greater than the interval threshold value among the pulse intervals in the time window is equal to or larger than the first number threshold value, the determining unit determines that there is a pulse interval reduction event.

In addition, the determining unit 118 calculates the reduction rate of the pulse amplitude (Step S108). Specifically, the determining unit 118 first compares an average amplitude with each of a plurality of pulse amplitudes obtained in the time window. The time window is the same as that used in the pulse interval reduction rate calculating process (Step S107). The average amplitude is the average value of the plurality of pulse amplitudes obtained in the second time range.

Then, the determining unit 118 counts the number of pulse amplitudes whose reduction rate that is determined by an amplitude difference, which is the difference between the two amplitudes, is equal to or larger than a predetermined amplitude threshold value. In this embodiment, it is assumed that the amplitude threshold value is, for example, 0.1. The reduction rate is obtained by the following Expression (3):

Reduction Rate=(Average Amplitude−Pulse Amplitude in Time Window)/Average Amplitude (3)

The determining unit 118 calculates the ratio of the counted number of pulse amplitudes in all the pulse amplitudes in the time window. When the ratio of the number of pulse amplitudes is equal to or larger than a second predetermined number threshold value, the determining unit 118 determines that there is a pulse amplitude reduction event. The second number threshold value indicates the ratio. The first number threshold value and the second number threshold value may be equal to or different from each other.

As described above, similarly to the pulse interval, it is known that the pulse amplitude is reduced when breathing is resumed during an apnea event. As such, it is possible to specify the resumption time of breathing by checking the reduction of the pulse amplitude.

As another example, when the counted number of pulse amplitudes, not the ratio of the number of pulse amplitudes, is equal to or larger than the second number threshold value, the determining unit may determine that there is a pulse amplitude reduction event. In this case, the second number threshold value indicates the number of pulse amplitudes.

As another example, the determining unit may count the number of pulse amplitudes in which the amplitude difference is equal to or larger than a predetermined amplitude threshold value, instead of calculating the reduction rate, and determine whether there is a pulse amplitude reduction event on the basis of the number of pulse amplitudes.

As another example, similarly to the pulse interval, when the number of successive pulse amplitudes greater than the amplitude threshold value among the pulse amplitudes in the time window is equal to or larger than the second number threshold value, the determining unit may determine that there is a pulse amplitude reduction event.

The interval threshold value and the amplitude threshold value may be determined by the standard deviation of data which is acquired for each subject when the subject is at rest.

The determining unit 118 calculates the ratio of the fluctuation variation component at the time (second time) immediately before the first time to the fluctuation variation component at each point of time (first time) (Step S109). In this embodiment, the second time is a predetermined time, for example, three seconds earlier than the first time.

As described above, it is known that the fluctuation variation component is reduced until just before during an apnea event breathing is resumed. Therefore, it is possible to specify the resumption time of breathing by checking for the reduction of the fluctuation variation component immediately before the first time.

The pulse interval reduction rate calculating process (Step S107), the pulse amplitude reduction rate calculating process (Step S108), and the fluctuation variation component reduction rate calculating process (Step S109) are independently performed and the order of the processes is not limited to this embodiment.

Then, when it is determined that there is a breathing interval reduction event or a breathing amplitude reduction event (Yes in Step S110), the determining unit 118 compares the reduction rate of the fluctuation variation component obtained in Step S109 with a predetermined fluctuation variation component threshold value, assuming the determined time that one of the two events occurs as the first time. The reduction rate of the fluctuation variation component is calculated by the following Expression (4), wherein it is assumed that the fluctuation variation component threshold value is, for example, 0.1:

Reduction Rate=(Average Value of Fluctuation variation component−Value of Fluctuation variation component in Time Window)/Average Value of Fluctuation variation component (4)

When the reduction rate of the fluctuation variation component obtained in Step S109 is equal to or larger than the fluctuation variation component threshold value, that is, when the fluctuation variation component is reduced immediately before the first time (Yes in Step S111), the determining unit 118 determines that an apnea event occurs (Step S112).

As described above, it is known that the fluctuation variation component is reduced immediately before breathing is resumed during an apnea event. Therefore, it is possible to determine whether a pulse interval reduction event or a pulse amplitude reduction event is caused by the apnea event by checking the reduction of the fluctuation variation component.

When it is determined that the ratio of the fluctuation variation component at the time (second time) with respect to the fluctuation variation component at the time (first time) during an apnea is small wherein the second time comes immediately before the first time and it is estimated that the breathing is resumed at the first time, it is considered to be an occurrence of a reduction in the fluctuation variation component immediately before the resumption of breathing. Therefore, in the pulse measuring device 10 according to this embodiment, when there is a breathing interval reduction event or a breathing amplitude reduction event and, at the same time, the ratio of the fluctuation variation component is less than a determined value, it is determined that an apnea event occurs.

On the other hand, when it is determined in Step S110 that neither the breathing interval reduction event nor the breathing amplitude reduction event occurs (No in Step S110) and when it is determined in Step S111 that the reduction rate of the fluctuation variation component is greater than the fluctuation variation component threshold value (No in Step S111), the process ends. In this way, the pulse measuring process of the pulse measuring device 10 is completed. When the number of apnea events obtained in this way is counted and the count number is equal to or larger than a threshold value, it is determined that there is a sleep apnea syndrome.

As described above, according to the pulse measuring device 10 of the embodiment, a reduction in the fluctuation variation component which appears immediately before breathing is resumed is considered as well as a reduction in the breathing interval or a reduction in the breathing amplitude when breathing is resumed during an apnea event. Therefore, it is possible to determine the presence or absence of an apnea event with high accuracy.

As a first modification to the first embodiment, in order to determine a reduction in the fluctuation variation component immediately before breathing is resumed during an apnea event, the determining unit 118 may calculate a gradient corresponding to a variation in the fluctuation variation component within the time range before the first time as a reference. When the gradient is less than a predetermined gradient threshold value, for example, “−10”, the determining unit 118 may determine that the fluctuation variation component is reduced immediately before the first time. The time range is, for example, equivalent to a time length for which the stop of breathing is maintained. As such, a reduction in the fluctuation variation component may be detected from the gradient of the fluctuation variation component in a predetermined time range immediately before a target time.

As a second modification, when the value of the fluctuation variation component at the time immediately before the time when an event of pulse interval reduction or an event of pulse amplitude reduction occurs is equal to or less than a predetermined fluctuation variation component threshold value, the determining unit 118 may determine that there is an apnea event.

As a third modification, the determining unit may determine whether there is a pulse interval reduction event by using pattern matching. Specifically, a typical pulse interval pattern in a predetermined time range when breathing is resumed during an apnea event is stored as a reference waveform (template) in the storage unit 120 in advance. The determining unit 118 extracts pulse interval data with a time length equal to the time range of the template out of the pulse interval data calculated by the interval calculating unit 114, and calculates the correlation between two pulse interval data items. Then, when the correlation between the two pulse interval data items is greater than a predetermined correlation threshold value, the determining unit determines that there is a pulse interval reduction event.

As a fourth modification, similarly to the third modification, the determining unit may determine whether there is a pulse amplitude reduction event by using pattern matching. Specifically, a typical pulse amplitude pattern in a predetermined time range when breathing is resumed during an apnea event is stored as a reference waveform (template) in the storage unit 120 in advance. The determining unit 118 extracts pulse amplitude data with a time length equal to the time range of the template from the pulse amplitude data calculated by the amplitude calculating unit 112, and calculates the correlation between two pulse amplitude data items. Then, when the correlation between the two pulse amplitude data items is greater than a predetermined correlation threshold value, the determining unit determines that there is a pulse amplitude reduction event.

As a fifth modification, the pulse measuring device 10, may separately determine an apnea event and a hypopnea event. While apnea is the complete stop of breathing, hypopnea is a state in which a person has difficulty in breathing and is less serious than apnea. In the determination of whether there is a hypopnea event, similar to the determination of whether there is an apnea event, when the pulse interval or the pulse amplitude is reduced and the fluctuation variation component is reduced, the determining unit may determine that there is a hypopnea event. However, as illustrated in FIG. 6, in the apnea and the hypopnea, the degrees of reduction in the pulse interval, the pulse amplitude, and the fluctuation variation component are different from each other.

The determining unit 118 stores the first interval threshold value, the first amplitude threshold value, and the first fluctuation variation component threshold value for determining whether there is an apnea event; and the second interval threshold value, the second amplitude threshold value, and the second fluctuation variation component threshold value for determining whether there is a hypopnea event in the storage unit 120. The determining unit 118 then separately determines whether there is an apnea event and there is a hypopnea event using each of the threshold values. The second interval threshold value is less than the first interval threshold value. The second amplitude threshold value is less than the second amplitude threshold value. The second fluctuation variation component threshold value is less than the first fluctuation variation component threshold value.

Specifically, first, a pulse wave measuring process illustrated in FIG. 4 is performed to determine whether there is an apnea event by using the first interval threshold value, the first amplitude threshold value, and the first fluctuation variation component threshold value. When it is determined that there is no apnea event, that is, when it is determined in Step S110 that neither the breathing interval reduction event nor the breathing amplitude reduction event occurs (No in Step S110), or when it is determined in Step S111 that the reduction rate of the fluctuation variation component is less than the first fluctuation variation component threshold value (No in Step S111), Step S110 and Step S111 are performed to determine whether there is a hypopnea event by using the second interval threshold value, the second amplitude threshold value, and the second fluctuation variation component threshold value.

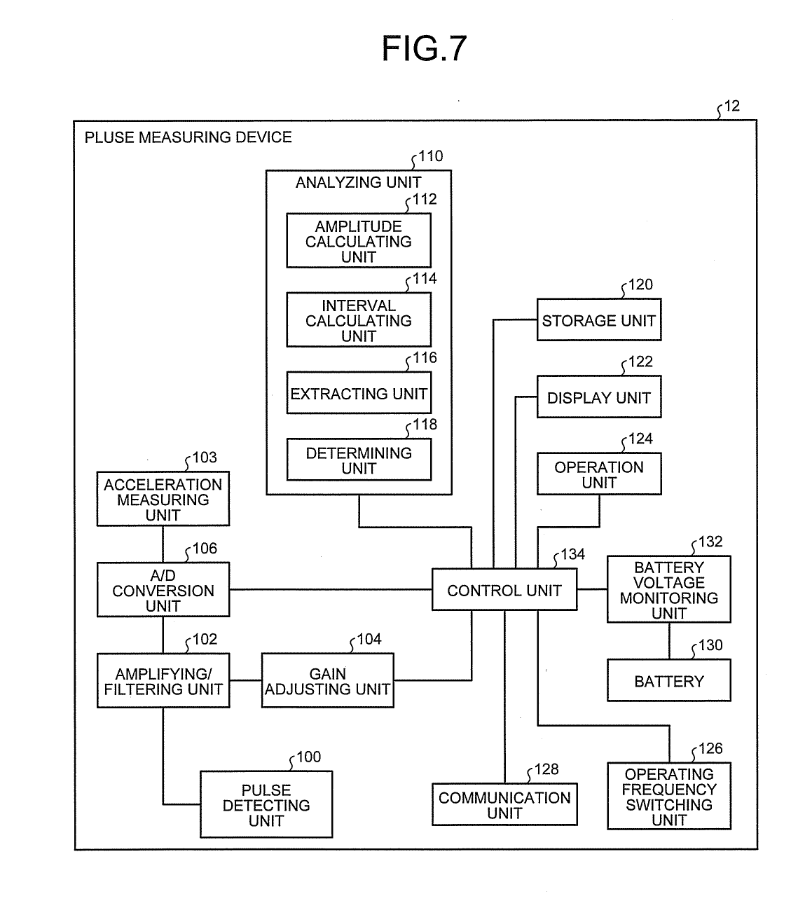

Next, a pulse measuring device 12 according to a second embodiment will be described. As illustrated in FIG. 7, the pulse measuring device 12 further includes an acceleration measuring unit 103 to measure a body motion at the same time as the measurement of a pulse wave. The acceleration measuring unit 103 measures the body motion of the subject.

When it is determined that the subject moves during the reduction of the pulse interval and the reduction of the pulse amplitude, the body motion may be considered as a reaction to the resumption of breathing. Considering into the measurement result of the body motion, it is possible to improve the accuracy of the determination of whether there is an apnea event. Specifically, in the event that the pulse interval and the pulse amplitude are reduced, there is a body motion, and the fluctuation variation component is reduced, the determining unit 118 determines that there is an apnea event. In this case, the amount of body motion is less than 0.1 g so as to exclude error data.

When the amount of body motion is large, the detection accuracy of the pulse interval is significantly reduced. Therefore, when a relatively large body motion is detected, the previous pulse interval is removed and Step S107 and the subsequent steps are performed. Specifically, when the amount of body motion is greater than 0.1 g after the detection of the pulse interval and the pulse amplitude, and during the detection of the pulse interval, at the time when the intersection point is detected, and for a predetermined period of time (for example, 0.3 seconds) from the detection of the intersection point; the determining unit 118 determines that the determination process is affected by the body motion and determines the detection data to be an error. Here, it is assumed that the time average value or the maximum value of the square-root of sum of squares of a variation in triaxial acceleration during sampling is the amount of body motion.

As described above, the pulse measuring device 12 can determine whether there is an apnea event with high accuracy by considering into the body motion.

The pulse measuring device according to the embodiment can determine an apnea event with high accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirits of the inventions.

What is claimed is:

1. A pulse measuring device comprising:
a pulse detecting unit configured to detect a pulse of a subject;
an amplitude calculating unit configured to calculate an amplitude of the pulse;
an interval calculating unit configured to calculate an interval of the pulses;
an extracting unit configured to extract a fluctuation variation component which varies in operative association with breathing of the subject from the interval; and
a determining unit configured to determine that an apnea event occurs when the amplitude or the interval is reduced at a first time and the fluctuation variation component is reduced at a second time which is immediately before the first time.

2. The device according to claim 1,
wherein the determining unit determines that the fluctuation variation component is reduced at the second time, when determining a ratio of a reduction in the fluctuation variation component at the second time with respect to the fluctuation variation component at the first time is equal to or larger than a predetermined first threshold value.

3. The device according to claim 2,
wherein the determining unit
compares each of a plurality of the intervals in a first time range including the first time with an average interval of a plurality of the intervals in a second time range that is before the first time range, and
determines that the interval is reduced when number of the intervals, in which a difference between the average interval and the interval is equal to or larger than a predetermined second threshold value, is equal to or larger than a predetermined third threshold value.

4. The device according to claim 3,
wherein the determining unit
compares each of a plurality of the amplitudes in the first time range with an average amplitude of a plurality of the amplitudes in the second time range and
determines that the amplitude is reduced when number of the amplitudes, in which a difference between the average amplitude and the amplitude is equal to or larger than a predetermined fourth threshold value, is equal to or larger than a predetermined fifth threshold value.

5. The device according to claim 4,
wherein
the determining unit
determines that the apnea event occurs when
the number of the intervals, in which the interval difference is equal to or larger than the second threshold value, is equal to or larger than the third threshold value, or the number of the amplitudes, in which the amplitude difference is equal to or larger than the fourth threshold value, is equal to or larger than the fifth threshold value, and
the ratio of the reduction in the fluctuation variation component is equal to or larger than the first threshold value, and
the determining unit determines that a hypopnea event occurs when
it is not determined that the apnea event occurs,
number of the intervals, in which the interval difference is equal to or larger than a predetermined sixth threshold value that is less than the second threshold value, is equal to or larger than the third threshold value or the number of the amplitudes, in which the amplitude difference is equal to or larger than a predetermined seventh threshold value that is less than the fourth threshold value, is equal to or larger than the fifth threshold value, and
the ratio of the reduction in the fluctuation variation component is equal to or larger than a predetermined eighth threshold value that is less than the first threshold value.

6. A pulse measuring method comprising:
detecting a pulse of a subject;
calculating an amplitude of the pulse;
calculating an interval of the pulses;
extracting a fluctuation variation component which varies in operative association with breathing of the subject from the interval; and
determining that an apnea event occurs when the amplitude or the interval is reduced at a first time and the fluctuation variation component is reduced at a second time which is immediately before the first time.
